(19) United States
(12) Patent Application Publication
(10) Pub. No.: US 2007/0259941 A1
(43) Pub. Date: Nov. 8, 2007

(54) Ramipril formulation

(75) Inventors: Paul Jonathan Harrison, Dublin (IE); Anna Marie Power, Dublin (IE)

Correspondence Address: STERNE, KESSLER, GOLDSTEIN & FOX P.L.L.C., 1100 NEW YORK AVENUE, N.W., WASHINGTON, DC, 20005, US

(73) Assignee: Selamine Limited, Dublin (IE)

(21) Appl. No.: 11/273,575

(22) Filed: Nov. 15, 2005

(30) Foreign Application Priority Data
Oct. 28, 2005 (GB) 0522047.0

(51) Int. Cl. A61K 31/40; A61P 9/10; C07D 207/04

(52) U.S. Cl. 514/412; 548/401; 548/452

(57) ABSTRACT

A Ramipril formulation which is suitably stabilised to control the degradation to the active metabolite ramiprilat.

### FIELD OF INVENTION

The present invention relates to a dosage form of Ramipril and also to methods of use. In particular, although not exclusively, the present invention relates to stability of formulations for treating or preventing various disease states involving the administration of Ramipril.

### BACKGROUND OF THE INVENTION

Ramipril, the United States Adopted Name (USAN) for (2S,3aS,6aS)-1[(S)-N-[(S)-1-carboxy-3-phenylpropyl]alanyl]octahydrocyclopenta[b]pyrrole-2-carboxylic acid, 1-ethyl ester (CAS Number 087333-19-5) is an angiotensin converting enzyme (ACE) inhibitor having the chemical structure shown below (I).

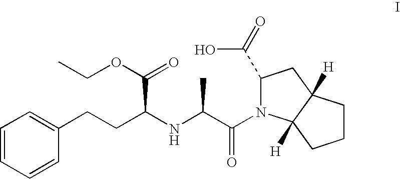

Ramipril and its acid are taught in EP 0 097 022. Ramipril has been used for the treatment of hypertension, heart failure, stroke, myocardial infarction, diabetes and cardiovascular disease. Ramipril may also reduce the risk of further strokes, heart attacks and cognitive impairment among stroke patients.

Ramipril is defined in official monographs in both the United States Pharmacopeia and the European Pharmacopoiea. In the European Pharmacopoiea 14 impurities are catagorised and labelled as impurities A-N. Impurities A, B, C and D are defined as qualified impurities with impurities E to N being classed as ‘other detectable impurities’. Different limits have been applied to the two sets of impurities. To fulfil the United States standard, only impurities A, B, C and D require quantification. Of the 14 impurities that are named in the European Pharmacopoiea only two are identified as potential degradation products: impurities D and E.

Impurity D, ramipril diketopiperazine, is not active as an ACE inhibitor whereas impurity E, ramipril diacid or ramiprilat, is up to 6 times more potent as an ACE inhibit than the parent compound ramipril. Ramipril is converted in vivo to ramiprilat and can therefore be considered to be a prodrug of ramiprilat.

Various Ramipril formulations are known in the art. Such formulations can be found in, for example, U.S. Pat. No. 4,743,450, U.S. Pat. No. 6,555,551, US 2005/0169981, WO 04/04809, US 2005/0069586, US 2003/0215526, WO 05/041940, and WO 03/059388. The present application does not concern these art known formulations.

Degradation of pharmaceutically active compounds is of concern to both medical practitioners and to the community at large. If significant degradation takes place between manufacture and administration of an active then suboptimal dosing is highly likely. For actives used in the treatment of hypertension and cardiovascular disease dosing accuracy is of tantamount importance as ineffective treatment is likely to result in life-threatening complications.

It would be useful if there were a formulation of Ramipril that avoids significant degradation to inactive impurities.

It is an object of the invention to overcome the disadvantages associated with present ramipril formulations or to at least provide the public with a useful alternative.

### SUMMARY OF THE INVENTION

Accordingly, in a first aspect the present invention provides a Ramipril formulation which is basic.

Formulation means all types of dosage forms that could be used for the oral administration of ramipril. Examples of such dosage forms include suspensions, solutions, tablets (chewable, dispersible and conventional), capsule formulations, multiparticulate formulations and formulations adapted to control the release of the drug from the oral dosage form, a so called sustained release formulation.

Solid formulations according to the invention give a pH of greater than 7 when made up as a 1% solution in water. Any formulations having this property are said to be basic. Liquid formulations according to the invention have a pH greater than 7.

Surprisingly it has been found that formulations which are basic undergo degradation in a different manner from those formulations presently known, ie acidic or neutral formulations. The preferred degradation pathway of basic formulations results in ramiprilat whereas other formulations result in the formation of inactive products such as ramipril diketopiperazine.

The altered degradation pathway is beneficial in the case of ramipril formulations because the product of the altered degradation pathway is an active metabolite of the drug. Degradation over time to other (inactive) products can thus be minimised.

The invention provides Ramipril formulations that display altered degradation pathway to the active metabolite ramiprilat, rather that the inactive diketopiperazine.

The “altered degradation pathway” is obtained by the inclusion of stabilisers in the formulation that makes the pH of a 1% solution in water basic in pH, .i.e greater than pH 7.

Preferred formulations according to the invention give a pH of greater than 7.5, more preferably greater than pH 8.

Liquid formulations according to the invention preferably have a pH of greater than 7.5, more preferably greater than pH 8.

The term “stabiliser” means any material that by its inclusion will render the pH of a 1% solution of the formulation basic. The examples of such “stabilisers” include carbonate salts, amino acids with basic side chains, and amines, although many suitable “stabilisers” will be know to those of skill in the art.

Preferred formulations according to the invention include citrate, carbonate salts, arginine, and ethanolamine, ethanolamine being particularly useful for liquid formations. Other examples of “stabilisers” include sodium lauryl sulphate, talc, magnesium stearate, sodium carbonate, sodium bicarbonate, calcium carbonate and salts.

In a further aspect the present invention also relates to a ramipril formulation that demonstrates substantially no degradation to ramipril diketopiperazine during storage. In preferred embodiments substantially all degradation taking place during storage is to ramiprilat.

The formulations of the invention may contain any suitable pharmaceutical excipients such as binders, coatings, sweeteners, surfactants, lubricants, glidants, fillers, other active ingredients, colorants and any other excipients or additives known to those in the art.

Formulations of the invention may contain buffers that keep the pH of the formulation within an alkaline range even in the presence of significant amounts of acid.

The formulations of the invention help to ensure that patients treated using said formulations receive the dose of ramipril (or ramiprilat) intended by the prescribing physician.

Formulations according to the invention also offer extended shelf lives. Because the efficacy of treatment does not decrease as the formulations of the invention age (or at least decreases at a vastly reduced rate when compared to known formulations) less wastage of expired medicaments occurs. There is therefore a concomitant reduction in unit cost for medicaments according to the invention over previously known formulations.

Preferred formulations according to invention give degradation to ramipril diketopiperazine during storage at 25° C. and 60% RH for 3 months of less than 1%, more preferably less than 0.5%.

Further preferred formulations according to invention give degradation to ramipril diketopiperazine during storage at 40° C. and 75% RH for 3 months of less than 4%, more preferably less than 2%.

In a further aspect the present invention also provides a method for treating or preventing a disease in a mammal selected from the group consisting of hypertension, heart failure, stroke, myocardial infarction, diabetes and cardiovascular disease or for reducing the risk of further strokes, heart attacks and cognitive impairment among stroke patients comprising administering to a mammal in need of such treatment a formulation according to the present invention.

In some embodiments the mammal is a non-human animal.

The present invention also provides the use of a formulation according to the present invention in the manufacture of a medicament for the treatment of hypertension, heart failure, stroke, myocardial infarction, diabetes and cardiovascular disease or for reducing the risk of further strokes, heart attacks and cognitive impairment among stroke patients.

In preferred embodiments the medicament is in the form of a capsule or tablet. However other embodiments include liquid formulations such as suspensions and syrups.

In a further aspect, this invention provides a therapeutic package suitable for commercial sale, comprising a container, a Ramipril formulation according to the invention, and, associated with said container, notice advising of extended shelf life.

For purposes of this invention Ramipril may be administered alone or in combination with other therapeutic agents. In one embodiment Ramipril is co-administered with a diuretic agent, preferably the diuretic is selected from hydrochlorothiazide or piretanide.

Ramipril is typically present in formulations according to the invention in an amount of from about 1.25 mg to about 10 mg. Other formulations may have 2.5 mg or 5 mg per tablet. The amount of active can be adjusted to be outside these limits depending, for example, on the size of the animal subject being treated (e.g., a horse). The term ‘Ramipril’ includes all the pharmaceutically acceptable versions thereof, e.g. salts, esters, clathrates thereof, and also anhydrous as well as hydrated forms.

In another aspect the invention provides a method for the manufacture of a ramipril formulation including the step of adding at least one basic compound. Basic compounds are known to those of skill in the art and suitable examples are included in the examples as well in this specification. The invention includes within its scope the manufacture of ramipril formulations using any suitable basic compound.

Various aspects of the invention will now be described with reference to examples.

### EXAMPLES

The following examples are provided to illustrate the invention only and should not be construed as limiting the scope of the invention as claimed herein. Some of the Example formulations set out herein fall within the scope of the invention as claimed.

The formulations herein may be varied, that is additions and replacement of ingredients with equivalents may be made, without departing from the scope of the invention as herein claimed. For example, the formulation mentioned may advantageously contain citrate salts in place of carbonates and bicarbonates whilst retaining the extended shelf life.

The examples presented focus on the lowest commercial strength, the 1.25 mg, where the highest percentage degradation would be expected (as %w/w with respect to dose). Those skilled in the art could easily formulate the higher strength products by adjusting the ratio of the stabiliser to drug substance to minimise the degradation of the drug substance and adjust the pathway so that the active metabolite was produced.

When ramipril (1.25mg) is simply mixed with the inert substance starch 130mg and stored in bottles for 1 month at 40° C. 75% Relative humidity, the drug degrades, and approximately 6% of impurity D is recorded. The pH of such a mixture is pH 5.25. With the inclusion of the base excipients it is possible to reduce the level of the impurity D and if used at increased levels convert the principle degradation product to impurity E ramiprilat as illustrated in the examples.

| | Formulation Reference | | | | | | | |
|---|---|---|---|---|---|---|---|---|
| | 1 | 2 | 3 | 4 | 5 | 6 | 7 | 8 |
| Ramipril | 1.25 | 1.25 | 1.25 | 1.25 | 1.25 | 1.25 | 1.25 | 1.25 |
| Sodium hydrogen | 0.3 | 0.6 | 0.9 | 1.25 | — | 1.00 | — | 0.83 |
| carbonate | | | | | | | | |
| Sodium carbonate | — | — | — | — | — | — | 0.625 | — |
| Calcium carbonate | — | — | — | — | — | — | — | 72.9 |
| Microcrystalline | — | — | — | — | — | — | 46.00 | — |
| cellulose | | | | | | | | |
| Calcium phosphate | — | — | — | — | — | 100.0 | — | — |
| dihydrate | | | | | | | | |
| Povidone k29/32 | — | — | — | — | — | — | — | 0.67 |
| Sodium starch | — | — | — | — | — | — | — | 4.17 |
| glycollate | | | | | | | | |
| Sodium lauryl sulphate | — | — | — | — | — | 0.5 | — | — |
| 1-Arginine | — | — | — | — | 0.9 | — | — | — |
| Calcium sulphate | 114.2 | 114.2 | 114.2 | 114.2 | 114.2 | — | — | — |
| Anhydrous lactose | — | — | — | — | — | — | 40.00 | — |
| Starch pregelatinised | 13.00 | 13.00 | 13.00 | 13.00 | 13.00 | — | — | — |
| L-HPC | — | — | — | — | — | 4.0 | — | — |
| Potato starch | — | — | — | — | — | — | 23.00 | — |
| Maize starch | — | — | — | — | — | 15.0 | — | — |
| Iron oxide red | 0.13 | 0.13 | 0.13 | | 0.13 | — | — | — |
| Silicon dioxide | — | — | — | — | — | — | 0.4 | — |
| Ethanol/water 1:1 | (q.s) | (q.s) | (q.s) | (q.s) | (q.s) | — | — | — |
| Water | | | | | | q.s | — | — |
| Talc | — | — | — | — | — | — | — | 2.09 |
| Sodium stearyl | 1.30 | 1.30 | 1.30 | 1.30 | 1.30 | | — | — |
| fumarate | | | | | | | | |
| Magnesium stearate | — | — | — | — | — | 1.30 | 1.30 | 0.83 |

Condition 40° C. 75% RH 14 days

| | | | | | | | | |
|---|---|---|---|---|---|---|---|---|
| Impurity D | 25% | 6.5% | 0.75% | 0.3%* | 0.48% | 0.49% | 0.36% | 0.11% |
| Impurity E | 2.5% | 2.1% | 1.1% | 2.0%* | 0.50% | 0.51% | 0.15% | 5.6% |
| pH 1% | 6.94 | 7.36 | 7.75 | 8.79 | 7.87 | 8.26 | 8.07 | 9.19 |

*1 month

The impurity levels reported in the examples attached are the levels of impurity when stored in bottles for 14 days at 40° C. 75 % relative humidity, with the exception of formulation 4 which was stored for 1 month at the same conditions.

All examples were manufactured on a small scale conventionally either by simply screening and blending the ingredients and then compressing, or if water or water ethanol mixture was used, screening, mixing, granulating drying in fluid bed drier, screening blending and compressing. These two processes direct blending and granulating and blending can be considered to be conventional granulation.

Preferably wet granulation is used to formulate basic formulations according to the invention to ensure that the principle degradation product is ramiprilat. Without wishing to be bound by theory, it is believed that wet granulation intimately mixes the ramipril with the basic compound offering better protection to the active compound from other ingredients which may catalyse degradation to the diketopiperazine.

The invention thus provides Ramipril-containing formulations where a stabiliser has been added that imparts an alkali pH to the formulation. The principle degradation product is ramiprilat.

1. A Ramipril formulation which is basic.

2. The formulation of claim 1 including a stabiliser selected from the group consisting of carbonate salts, amino acids with basic side chains, and amines.

3. The formulation of claim 1 wherein the stabliser is selected from the group consisting of arginine, ethanolamine, sodium lauryl sulphate, talc, magnesium staerate, sodium carbonate, sodium bicarbonate, calcium carbonate and citrate salts.

4. The formulation of claim 1 wherein the pH of the formulation is greater than 7.5.

5. The formulation of claim 4 wherein the pH of the formulation is greater than 8.

6. The formulation of claim 1 wherein degradation to ramipril diketopiperazine during storage at 25° C. and 60% RH for 3 months is less than 1%.

7. The formulation of claim 1 wherein degradation to ramipril diketopiperazine during storage at 25° C. and 60% RH for 3 months is less than 0.5%.

8. The formulation of claim 1 wherein degradation to ramipril diketopiperazine during storage at 40° C. and 75% RH for 3 months is less than 4%.

9. The formulation of claim 1 wherein degradation to ramipril diketopiperazine during storage at 40° C. and 75% RH for 3 months is less than 2%.

10. A Ramipril formulations that displays a major degradation pathway to the active metabolite ramiprilat, rather that the inactive diketopiperazine.

11. The formulation of claim 10 wherein the major degradation pathway is obtained by the inclusion of stabilisers in the formulation that makes it basic.

12. The formulation of claim 11 including a stabiliser selected from the group consisting of carbonate salts, amino acids with basic side chains, and amines.

13. The formulation of claim 11 wherein the stabliser is selected from the group consisting of arginine, ethanolamine, sodium lauryl sulphate, talc, magnesium staerate, sodium carbonate, sodium bicarbonate, calcium carbonate and citrate salts.

14. The formulation of claim 10 wherein the formulation is in a liquid form and includes ethanolamine as a stabiliser.

15. The ramipril formulation of claim 10 wherein the formulation additionally comprises a diuretic.

16. The formulation of claim 10 wherein the pH of the formulation is greater than 7.5.

17. The formulation of claim 10 wherein the pH of the formulation is greater than 8.

18. A ramipril formulation that demonstrates substantially no degradation to ramipril diketopiperazine during storage.

19. The formulation of claim 18 wherein degradation to ramipril diketopiperazine during storage at 25° C. and 60% RH for 3 months is less than 1%.

20. The formulation of claim 18 wherein degradation to ramipril diketopiperazine during storage at 25° C. and 60% RH for 3 months is less than 0.5%.

21. The formulation of claim 18 wherein degradation to ramipril diketopiperazine during storage at 40° C. and 75% RH for 3 months is less than 4%.

22. The formulation of claim 18 wherein degradation to ramipril diketopiperazine during storage at 40° C. and 75% RH for 3 months is less than 2%.

23. A method for treating or preventing a disease in a mammal selected from the group consisting of hypertension, heart failure, stroke, myocardial infarction, diabetes and cardiovascular disease or for reducing the risk of further strokes, heart attacks and cognitive impairment among stroke patients comprising administering to a mammal in need of such treatment the formulation of 19.

24. A therapeutic package suitable for commercial sale, comprising a container, a basic Ramipril formulation, and, associated with said container, notice advising of extended shelf life.

25. A method for the manufacture of a ramipril formulation including the step of adding at least one basic compound.

26. The method of claim 25 wherein the basic compound is a citrate salt, L-arginine, or a carbonate salt.

27. The method of claim 26 wherein the basic compound is a citrate salt.

28. The method of claim 25 wherein the method additionally includes the step of wet granulation, dry granulation or direct compression.

29. The method of claim 25 wherein the method includes the step of wet granulation.
